(19) United States
(12) Patent Application Publication
(10) Pub. No.: US 2025/0214909 A1
(43) Pub. Date: Jul. 3, 2025

(54) CARRIER-BASED AGRICULTURAL BIOFERTILIZER COMPOSITION

(71) Applicants: BENOY BERRY, LONDON (GB); PRAVEEN KUMAR DIVVELA, WEST GODAVARI (IN)

(72) Inventors: BENOY BERRY, LONDON (GB); PRAVEEN KUMAR DIVVELA, WEST GODAVARI (IN)

(21) Appl. No.: 19/087,638

(22) Filed: Mar. 24, 2025

(30) Foreign Application Priority Data
Mar. 27, 2024 (GB) GB2404386.1

(51) Int. Cl. C05F 11/08; C05G 3/00; C05G 5/20; C12N 1/20; C12R 1/085

(52) U.S. Cl. CPC C05F 11/08; C05G 3/00; C05G 5/20; C12N 1/20; C12R 2001/085

(57) ABSTRACT

Exemplary embodiments of the present disclosure are directed towards the carrier-based agricultural biofertilizer composition, includes an isolated Bacillus Cereus strain CGAPGPBBS-048 having the deposit accession number KY495213; an agriculturally acceptable liquid formulation including a stabilizer, that may provide stability and support for at least one of the Bacillus species, thereby enhancing the growth and overall health of the plants.

### CROSS-REFERENCE TO RELATED APPLICATIONS

This patent application claims priority benefit of UK Patent Application No: GB2404386.1, entitled “A CARRIER-BASED AGRICULTURAL BIOFERTILIZER COMPOSITION”, filed on 27 Mar. 2024. The entire contents of the patent application are hereby incorporated by reference herein in its entirety.

### TECHNICAL FIELD

The present disclosure generally relates to the agricultural fertilizer compositions, more particularly the invention relates to a biofertilizer composition and a method for enhancing soil fertility using a carrier-based agricultural biofertilizer composition.

### BACKGROUND

Agricultural biofertilizers can benefit from ongoing developmental research to further enhance their effectiveness and contribute to sustainable agriculture. Here are some key areas of research that can support the development and optimization of agricultural biofertilizers:
a) Microbial diversity: Research can focus on understanding the diversity and functionality of beneficial microorganisms present in biofertilizers. By identifying specific strains or consortia of microorganisms that promote plant growth, nutrient uptake, and soil health, researchers can develop biofertilizers with improved performance.
b) Formulation and application methods: Studying the formulation and application methods of biofertilizers can help optimize their effectiveness. Research can explore different carriers or substrates to enhance microbial survival and activity during storage and application. Additionally, studies can investigate the optimal application rates, timings, and techniques for different crops and soil types.
c) Compatibility with other inputs: Research can assess the compatibility of biofertilizers with other agricultural inputs, such as synthetic fertilizers, pesticides, and biostimulants. Understanding the interactions between biofertilizers and these inputs can help farmers optimize their use and reduce potential negative interactions.
d) Long-term effects on soil health: Long-term studies are needed to evaluate the impact of biofertilizers on soil health and ecosystem services. Research can investigate their effects on soil organic matter content, microbial communities, soil structure, and nutrient cycling over multiple cropping seasons. This information can guide sustainable soil management practices and provide insights into the long-term benefits of biofertilizers.
e) Field trials and validation: Conducting field trials in different agroecosystems and regions is crucial for validating the performance of biofertilizers under real-world conditions. Research can evaluate the effectiveness of biofertilizers in terms of crop yield, nutrient content, pest and disease resistance, and overall sustainability. These trials can also help identify specific crops, soil types, and environmental conditions where biofertilizers benefit most.
f) Economic viability and scaling up: Research can explore biofertilizer production and distribution's economic viability and scalability. Studies on cost-effectiveness, market demand, and business models can help promote the adoption of biofertilizers by farmers and facilitate their integration into existing agricultural systems.

Plant growth-promoting Bacillus (PGPB) are beneficial bacteria that enhance plant growth and crop health. These Bacillus species colonize plant roots, stimulating root development and offering various benefits. They produce enzymes like phosphate solubilizing enzymes, breaking down soil-bound nutrients for plants. Bacillus species also fix atmospheric nitrogen, increasing nutrient availability for crops and improving growth. Certain strains inhibit plant pathogens through antibiotics and antimicrobial compounds, reducing disease reliance on chemical pesticides. Bacillus triggers systemic plant resistance, producing defense-related proteins and compounds against diseases, bolstering plant defenses during stresses like pathogen attacks, drought, and salinity. PGPB help plants tolerate abiotic stresses by producing stress-related enzymes and osmoprotectants. They enhance water and nutrient uptake efficiency, enhancing resilience in harsh conditions. Plant growth-promoting Bacillus enhances soil health by improving structure, nutrient cycling, and organic matter decomposition, boosting fertility and productivity. Such soils, with diverse microbial communities including PGPB, create favorable environments for plant growth, fostering sustainable agriculture. Strategies incorporating PGPB reduce chemical inputs, promote ecological balance, and enhance agricultural sustainability.

Conventional agriculture, reliant on chemical fertilizers, presents numerous drawbacks and limitations that affect both the environment and long-term sustainability. Poorly managed chemical fertilizers contribute to environmental pollution, with excess application leading to nutrient runoff into water bodies, causing water pollution and eutrophication. This depletes oxygen in aquatic ecosystems, harming aquatic life. Unchecked use of chemical fertilizers leads to soil degradation, lacking in practices for soil health improvement. This results in nutrient imbalances, reduced fertility, microorganism loss, and soil structure degradation, lowering crop productivity. Synthetic pesticides, often used in conventional agriculture, harm beneficial organisms, causing biodiversity loss. Reduced biodiversity disrupts ecological balance, affecting pest control and potentially promoting pest resistance. Chemical fertilizer production demands substantial energy from fossil fuels, worsening carbon emissions and climate change. Phosphate and potassium mining for fertilizers deplete finite resources. Prolonged exposure to these chemicals endangers farm workers' health, and residue contamination on crops poses risks to consumers. Conventional agriculture's heavy water use intensifies water scarcity, particularly in water-scarce regions, exacerbating resource depletion due to crop growth demands. To counter these issues and promote sustainability, alternative practices like organic farming, integrated pest management, and organic/bio-based fertilizers are adopted. These approaches minimize environmental harm, support soil health, biodiversity, and reduce reliance on non-renewable resources. They prioritize the long-term viability of agricultural systems.

In the light of the aforementioned discussion, there exists a need for a certain system with novel methodologies that would overcome the above-mentioned disadvantages.

### SUMMARY

The following presents a simplified summary of the disclosure in order to provide a basic understanding of the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

An embodiment of the present disclosure is directed towards carrier-based agricultural biofertilizer composition.

An embodiment of the present disclosure is directed towards the carrier-based agricultural biofertilizer composition, includes an isolated Bacillus Cereus strain CGAPGPBBS-048 having the deposit accession number KY495213; an agriculturally acceptable liquid formulation including a stabilizer, that may provide stability and support for at least one of the Bacillus species, thereby enhancing the growth and overall health of the plants.

An objective of the present disclosure is directed towards enabling augmented plant growth and bolstering overall plant health.

An objective of the present disclosure is directed towards endowing plant growth-promoting abilities by proficiently generating phytohormones, notably indoleacetic acid, alongside the secretion of siderophores and other secondary metabolites that confer protection against pathogenic organisms.

An objective of the present disclosure is directed towards the effective production of phytohormones, particularly indoleacetic acid, to heighten plant growth and elevate germination rates.

An objective of the present disclosure is directed towards the capacity to generate phytohormone, specifically indole acetic acid, resulting in enhanced plant germination and growth.

An objective of the present disclosure is directed towards promoting robust plant growth and enhancing overall plant health across diverse agricultural settings.

An objective of the present disclosure is directed towards harnessing the inherent potential of the Bacillus Cereus strain CGAPGPBBS-048 to produce essential phytohormones, particularly emphasizing the significance of indoleacetic acid in stimulating growth processes.

An objective of the present disclosure is directed towards maximizing plant health by leveraging the biofertilizer composition's capacity to secrete siderophores and secondary metabolites, offering effective protection against harmful pathogens that could impede plant growth.

An objective of the present disclosure is directed towards strategically generating indoleacetic acid through controlled application, thereby facilitating robust plant growth, especially during critical germination phases.

An objective of the present disclosure is directed towards offering an environmentally friendly approach to enhancing agricultural practices, empowering farmers to foster healthier plant growth and elevated yields without resorting to synthetic chemicals.

An objective of the present disclosure is directed towards enhancing crop resilience and successful cultivation even in challenging environments, achieved by optimizing indoleacetic acid synthesis and its positive impact on plant adaptation.

An objective of the present disclosure is directed towards providing a sustainable solution for agricultural advancement, reducing reliance on excessive chemical interventions and contributing to soil health preservation and ecological equilibrium.

An objective of the present disclosure is directed towards facilitating resource-efficient farming practices aligned with ecological conservation and long-term agricultural sustainability.

An objective of the present disclosure is directed towards improving the quality of harvested produce, translating into tangible benefits for growers and consumers alike.

An objective of the present disclosure is directed towards addressing the evolving needs of agriculture through innovative solutions, balancing productivity, environmental considerations, and economic viability by leveraging the growth-promoting attributes of the Bacillus strain.

An embodiment of the present disclosure directed towards a method for enhancing soil fertility using of a biofertilizer composition

Furthermore, the objects and advantages of this invention will become apparent from the following description and the accompanying annexed drawings.

### BRIEF DESCRIPTION OF THE DRAWINGS

In the following, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

FIG. 1 is a flowchart depicting a method for enhancing soil fertility using of a biofertilizer composition

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

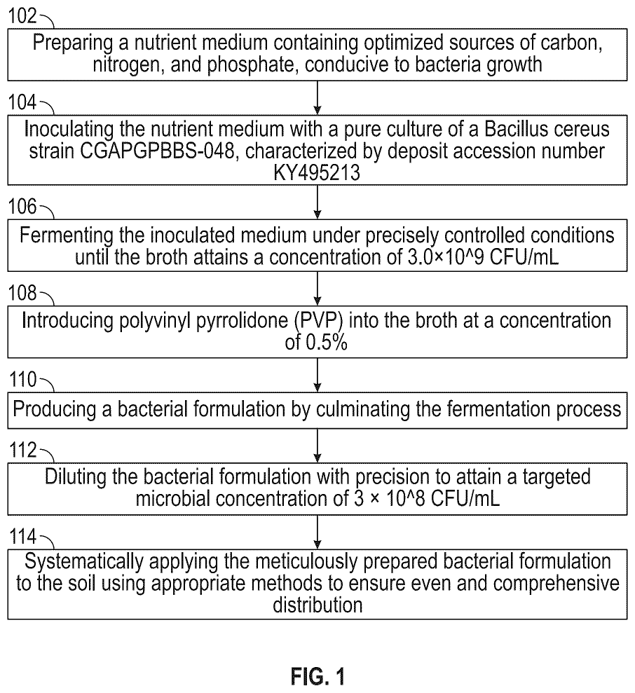

FIG. 1 is a flowchart depicting a method for enhancing soil fertility using a carrier-based agricultural biofertilizer composition. The method starts at step 102 by preparing a nutrient medium containing optimized sources of carbon, nitrogen, and phosphate, conducive to bacteria growth. At step 104, inoculating the nutrient medium with a pure culture of a Bacillus Cereus strain CGAPGPBBS-048, characterized by deposit accession number KY495213. Fermenting the inoculated medium under precisely controlled conditions until the broth attains a concentration of 3.0×10\hat{ }9 CFU/mL at step 106. Next at step 108, introducing polyvinyl pyrrolidone (PVP) into the broth at a concentration of 0.5%. At step 110, producing a bacterial formulation by culminating the fermentation process. Diluting the bacterial formulation with precision to attain a targeted microbial concentration of 3×10\hat{ }8 CFU/mL at step 112. Next at step 114, systematically applying the meticulously prepared bacterial formulation to the soil using appropriate methods to ensure even and comprehensive distribution.

In another exemplary embodiment, the nutrient medium may include carbon sources selected from the group consisting of sucrose and glucose, whereby the carbon sources may provide optimal conditions for bacterial growth and nutrient assimilation.

In another exemplary embodiment, the nutrient medium may include nitrogen sources selected from the group consisting of ammonium sulfate, urea, and peptone, whereby the nitrogen sources may provide optimal conditions for bacterial growth and nutrient assimilation.

In another exemplary embodiment, the step of fermenting conditions may carried out under controlled conditions including temperature, pH, and oxygen levels, to thereby promote optimal bacterial growth and metabolic activity.

In another exemplary embodiment, the step of systematically applying the meticulously prepared bacterial formulation to soil is performed using sprinkler irrigation, spray application, drone-based application, soil mixing techniques, seed deepening methods, plant root deepening methods, or any combination thereof.

In an exemplary embodiment, the carrier-based agricultural biofertilizer composition may include an isolated Bacillus Cereus strain CGAPGPBBS-048 having the deposit accession number KY495213; an agriculturally acceptable liquid formulation including a stabilizer, that may provide stability and support for at least one of the Bacillus species, thereby enhancing the growth and overall health of the plants.

Indole acetic acid, is a plant hormone that plays a crucial role in various aspects of plant growth and development, including cell elongation, root formation, fruit development, and responses to environmental stimuli. Some Bacillus spp. have the ability to produce IAA, and their interactions with plants can have significant positive effects on plant growth and yield. IAA-producing Bacillus strains are capable of synthesizing IAA through various pathways. They convert tryptophan, an amino acid present in the rhizosphere, into IAA using enzymes such as tryptophan-2-monooxygenase or indole-3-acetamide hydrolase. The synthesized IAA is released into the surrounding environment and can be taken up by plant roots. IAA-producing Bacillus strains enhance nutrient availability in the soil by solubilizing inorganic phosphates and fixing atmospheric nitrogen. It promotes root growth and development. The hormone stimulates cell division and elongation in the root system, resulting in a denser and more extensive root network. Increased root surface area improves nutrient and water uptake, leading to healthier and more vigorous plants. It can induce systemic resistance in plants. They activate plant defense mechanisms, such as the production of pathogenesis-related proteins and phytoalexins, to protect plants from pathogenic microorganisms. This enhanced resistance helps plants combat diseases and reduces the need for chemical pesticides.

The use of IAA-producing Bacillus strains for plant growth promotion offers an environmentally friendly alternative to synthetic fertilizers and pesticides. These bacteria enhance nutrient availability without causing environmental pollution and reduce the reliance on chemical inputs. They also contribute to soil health by improving its structure, nutrient cycling, and overall microbial diversity. IAA-producing Bacillus strains help plants cope with various abiotic stresses, such as drought, salinity, and heavy metal toxicity. The hormone regulates stomatal closure, reducing water loss during drought conditions. It also enhances the synthesis of osmoprotectants and antioxidant enzymes, which protect plants against salt stress and oxidative damage caused by heavy metals. Enhanced nutrient availability, root development, induced resistance, and stress tolerance increase plant yield and productivity. Plants inoculated with indole acetic acid-producing bacteria often exhibit higher biomass accumulation, increased fruit/seed production, and improved quality characteristics.

Alkaline soils and high pH cause many severe impacts on crop yield and quality. While indole acetic acid-producing bacteria have numerous benefits for plant growth and yield, they may face certain challenges when applied in alkaline soil conditions. Alkaline soil conditions can negatively affect the activity and survival of IAA-producing Bacillus strain. These bacteria generally thrive in neutral to slightly acidic soil environments. The high pH of alkaline soil can inhibit their growth, nutrient uptake, and metabolic activities, leading to a decrease in indole acetic acid production.

Alkaline soil conditions can disrupt the metabolic pathways of indole acetic acid synthesis in bacteria. The enzymes involved in tryptophan conversion to indole acetic acid may have reduced activity or be inhibited by the high pH, resulting in lower levels of hormone production. This can limit the beneficial effects of indole acetic acid on plant growth and development. Alkaline soil often has limited availability of certain essential nutrients, such as iron and manganese, due to their reduced solubility. Indole acetic acid-producing bacteria require these nutrients as cofactors for enzyme activity and hormone synthesis. In alkaline soil, these nutrients may be less accessible to both the bacteria and plants, affecting their growth-promoting effects.

Decreased root colonization of IAA-producing Bacillus strains. Indole acetic acid-producing bacteria establish a symbiotic relationship with plant roots, colonizing the rhizosphere and rhizoplane. However, alkaline soil conditions can impede the ability of these bacteria to colonize the roots effectively. The high pH can alter root exudation patterns, reducing the attraction and attachment of the bacteria to the root surface. This can limit the direct interaction between the bacteria and plant roots. Alkaline soil conditions can influence the sensitivity and response of plants to indole acetic acid. Higher soil pH can affect the perception and signaling of the hormone within plant cells, potentially reducing the plant's ability to utilize and respond to indole acetic acid produced by the bacteria. This can weaken the growth-promoting effects of the hormone on plant physiology. Alkaline soil environments often favor certain microbial populations that are adapted to these conditions. These indigenous alkaline-tolerant microorganisms may outcompete indole acetic acid-producing bacteria for resources and niche occupancy. As a result, the establishment and survival of these bacteria in alkaline soil can be compromised.

In an exemplary embodiment, an isolated Bacillus Cereus strain CGAPGPBBS-048 may be derived from the alkaline calcareous soil found in a cowpea farming field located in Yola, Nigeria. This strain may have been incorporated into a biofertilizer formulation designed to significantly enhance seed germination in soils with higher pH levels. The chosen strain demonstrates its efficacy in enhancing plant growth and promoting overall plant health by effectively producing IAA, specifically benefiting crops such as Cowpea (Vigna unguiculata), Groundnut (Arachis hypogaea), and Soybean (Glycine max) during both rainy and dry seasons.

Morphological Characteristics of the Bacillus Cereus strain CGAPGPBBS-048:
Cell shape: Spherical colonies,
Cell size: 2.5 mm
Cell arrangement: rod arrangement
Gram stain: Positive
Motility: Yes
Pigment: Absent
Capsule: Absent
Spores: Endospore formation

Physiological Properties of the Bacillus Cereus strain CGAPGPBBS-048:
Behavior to oxygen: Aerobic or facultative anaerobic
Conditions for growth: pH-6.5-8.5
Temperature −37±2° C.

In another exemplary embodiment, despite possessing valuable inherent traits, certain beneficial bacterial strains may not naturally inhabit a given field soil or, if present, could be limited in numbers or exhibit reduced activity. This may result in an inability to confer advantageous effects on plants within an unaltered or unenhanced rhizosphere.

In order for a bacterial strain with inherent beneficial traits, such as a plant growth-promoting rhizobacteria (PGPR), for a bacterium to have a positive impact on plant growth, it needs to exhibit competitive advantages and establish effective colonization within the rhizosphere during periods of active plant growth. It's crucial to acknowledge that, in the absence of interventions or enhancements, the probability of any indigenous or naturally occurring PGPR, like the bacterial strain CGAPGPBBS-048, conferring benefits to plant growth is rather limited. This emphasizes the importance of creating a conducive environment to facilitate the successful colonization and effectiveness of PGPR strains.

In another exemplary embodiment, The PGPR strain CGAPGPBBS-048, potentially isolated from the rhizosphere, exhibits a noteworthy trait of producing indole acetic acid under higher pH conditions, thereby contributing to enhanced plant growth. Moreover, the utilization of specialized laboratory cultivation techniques might have been employed to optimize the growth and population density of CGAPGPBBS-048, effectively harnessing its full PGPR potential. These carefully tailored growth conditions are likely identified to bolster the competitive edge of CGAPGPBBS-048 upon application to the rhizosphere, ultimately resulting in a favorable impact on plant growth. Essentially, the meticulous determination of growth parameters conducive to the prosperous colonization of CGAPGPBBS-048 within the rhizosphere has paved the way for its remarkable positive influence on plant growth, a feat unattainable under ordinary circumstances.

In another exemplary embodiment, a biologically pure culture of CGAPGPBBS-048 is cultivated to establish a stock culture, with portions of this culture meticulously preserved in cryogenic vials at a temperature of −80° C. When undertaking production runs, the frozen stock culture serves as the inoculum for a flask containing nutrient-rich broth media, all under carefully controlled and optimized conditions.

In another exemplary embodiment, the bacterial culture may undergo cultivation within a temperature spectrum of 30-32° C. during the rainy season or alternatively, within the range of 48-50° C. during the dry season, in various iterations. This flask culture may then magnify through scaling in a fermenter, maintaining akin growth conditions. This amplification may culminate in augmented population proliferation and an accentuated manifestation of plant growth-promoting attributes inherent to CGAPGPBBS-048, as the culture approaches the early stationary phase. Small quantities drawn from this culture at the early stationary phase are meticulously placed in sterilized plastic bags under aseptic conditions. The final product achieves a minimum concentration of the active ingredient, ensuring a viability of at least 3×10⁸ colony-forming units per mL. Strain CGAPGPBBS-048's fermented culture thrives under optimized conditions to optimize bacterial vitality and preserve plant growth-promoting characteristics. The resultant liquid formulation, securely encased in sterile bags, remains amenable to viable count analyses. Particularly noteworthy, CGAPGPBBS-048 responds distinctively to fluctuations in temperature when subjected to diverse thermal conditions. Examinations into its shelf life underscore its robustness. Even subsequent to an 18-month period of storage at both specified temperatures, the minimal count of bacterial cells steadfastly retains at 2×10⁸, thereby underlining the strain's enduring viability over time.

In another exemplary embodiment, the potential phytotoxic effects of Bacillus Cereus strain CGAPGPBBS-048 were evaluated through a field experiment, where symptoms including tip injury, wilting, vein clearing, necrosis, and epinasty/hyponasty were closely monitored. Intriguingly, no such symptoms were observed either during or following the application of Bacillus Cereus CGAPGPBBS-048 across all three crops during both seasons. These compelling observations suggest the absence of any deleterious impact on the plants, underscoring CGAPGPBBS-048 efficacy as a plant growth-promoting bacterium suitable for commercial utilization under both regular and elevated pH conditions. Moreover, its versatility extends to various temperature conditions within the Nigerian context.

In another exemplary embodiment, the Bacillus Cereus strain CGAPGPBBS-048 may capable of producing indole acetic acid in higher pH conditions by expressing a gene encoding an indole acetic acid-producing enzyme.

In another exemplary embodiment, wherein the Bacillus Cereus strain CGAPGPBBS-048 may be isolated from rhizosphere soil of Yola, Nigeria.

In another exemplary embodiment, wherein the Bacillus Cereus strain CGAPGPBBS-048 may be isolated by a serial dilution technique.

In another exemplary embodiment, the Bacillus Cereus strain CGAPGPBBS-048 may has the ability to stimulate plant growth through the production of phytohormones, including indoleacetic acid, thereby regulating plant growth, improving root development, and enhancing overall plant vigor.

Amplification and 16S rRNA Gene Sequence Analysis: After amplifying the partial 16S rRNA gene, cycle sequencing was performed through MACROGEN in Korea. The resulting amplified product underwent sequencing using the forward sequencing reaction mix. To determine homology, the DNA sequence was searched using the BLAST search engine at the NCBI site (ncbi.nlm.nih.gov) and FASTA (ebi.ac.uk). The FASTA homology search revealed similarity to Bacillus megaterium, and the corresponding strain obtained from NCBI was designated as KY495213.

### Example: 1—Isolation of Rhizobacteria: Bacteria were Isolated from the Alkaline Calcareous Soil of Rhizosphere and Rhizoplane from Yola, Nigeria by a Serial Dilution Technique and Spread Plate Method

Process for isolation and cultivation of Bacteria from Soil Sample:
a) Obtain a sample containing bacteria, either by scraping the soil surface or using a sterile instrument to collect a soil core.
b) Prepare nutrient agar plates by sterilizing them through autoclaving and allowing them to cool to room temperature. These plates will serve the purpose of isolating bacteria from the soil sample.
c) Take a small portion of the soil sample and evenly distribute it across the surface of the nutrient agar plate. Incubate the plates at temperatures ranging from 35-37° C. for a period of 24-48 hours.
d) After the incubation period, bacterial growth should be visible on the nutrient agar plates. Select one or more colonies and streak them onto new nutrient agar plates to obtain pure cultures.
e) Store the pure cultures in 50% glycerol solution for future utilization.

### Example: 2—Indole Acetic Acid Test: Indole Acetic Acid Test Steps of Bacterial Strain CGAPGPBBS-048

i. The peptone water was supplemented with tryptophan to prepare indole-producing media.
ii. Select a pure culture of the bacterial strain, and transfer a loopful of the bacterial culture into the corresponding test tubes.
iii. Incubate the inoculated test tubes at 37° C. for 24-48 hours.
iv. add approximately 0.5 mL of Kovacs reagent to detect indole acetic acid to each test tube.
v. Observe the test tubes for the appearance of red color in the Kovacs reagent layer at the top of the broth, and take the optical density (OD) at 530 nm.

### Example: 3—Indole Acetic Acid Test in a Higher pH Medium

a. Prepare nutrient agar plates and streak the bacterial strain CGAPGPBBS-048 for fresh culture. Incubate the plates at the appropriate temperature for 24-48 hours until visible colonies form.
b. Inoculate a single colony of the bacterial strain CGAPGPBBS-048 into a 5 mL trypticase soya broth and incubate at the appropriate temperature with shaking for 24 hours.
c. Prepare a higher pH medium using Tris-HCl buffer in the peptone water was supplemented with tryptophan to prepare indole-producing media (adjust the pH up to 8.5).
d. The bacterial isolate (0.3 ml inoculum with approximately 10⁸ CFU ml⁻¹) was inoculated into a test tube containing 10 mL of the low pH medium (peptone water with tryptophan). Incubate the test tube at the appropriate temperature with shaking.
e. Incubate the inoculated test tubes at 37° C. for 24-48 hours.
f. Add approximately 0.5 mL of Kovacs reagent to detect indole acetic acid to each test tube.
g. Observe the test tubes for the appearance of red color in the Kovacs reagent layer at the top of the broth, and take the optical density (OD) at 530 nm.

TABLE 1
Indole acetic acid production in a higher pH medium

| Bacillus Cereus CGAPGPBBS-048 | | | | Escherichia coli | | |
|---|---|---|---|---|---|---|
| | Optical | | | Optical | Indole | |
| Time | Density | Indole acetic | pH of | Density | acetic acid | pH of |
| (days) | (660 nm) | acid (mg L⁻¹ ) | medium | (660 nm) | (mg L⁻¹ ) | medium |
| 1 | 1.5 ± 0.02ef | 120.09 ± 1.40f | 8.31 ± 0.10a | 0.5 ± 0.01e | 15.85 ± 0.18f | 8.67 ± 0.01a |
| 2 | 1.64 ± 0.02d | 126.09 ± 1.47f | 7.8 ± 0.09b | 0.6 ± 0.01d | 19.81 ± 0.23e | 8.6 ± 0.06a |
| 3 | 1.79 ± 0.02bc | 136.1 ± 1.59f | 7.39 ± 0.09b | 0.99 ± 0.01a | 25.76 ± 0.30d | 8.52 ± 0.10ab |
| 4 | 1.94 ± 0.11a | 154 ± 8.48e | 6.42 ± 0.35c | 0.89 ± 0.05b | 28.46 ± 1.57d | 8.31 ± 0.27b |
| 5 | 1.91 ± 0.10ab | 170.42 ± 9.39d | 5.79 ± 0.32d | 0.77 ± 0.04c | 32.52 ± 1.79c | 7.83 ± 0.20c |

### Example: 4—Inoculation Effect on Cowpea, Groundnut, and Soybean Seed Emergence

a) Bacterial strain CGAPGPBBS-048 was grown in 100 ml of Tryptic soy broth for 48 hours on a rotary shaker. Bacterial cell culture was concentrated by centrifugation and washed with distilled water, and suspended in NaCl solution.
b) Take the surface sterilized legume seeds and place the legume seeds in a petri dish lined with a damp paper towel. Ensure that the seeds are spread out evenly and not touching each other.
c) Add the appropriate amount of inoculant to the seeds in the petri dish. Mix the seeds and inoculant gently to ensure that the seeds are evenly coated.
d) Seal the petri dish with parafilm to maintain moisture and prevent contamination.
e) Place the petri dish in a warm location (30° C.) for 24-48 hours to allow for the inoculant to adhere to the seeds.
f) After the incubation period, plant the inoculated seeds in sterile soil with 3 cm planting depth and spacing for the specific legume species.
g) Water the seeds immediately after planting and ensure that the soil remains moist throughout the germination period.
h) Record the number of seedlings that emerge from the inoculated seeds and compare it to the number of seedlings that emerge from non-inoculated seeds.
i) Calculate the percentage of seedling emergence for both the inoculated and non-inoculated seeds.
j) Analyze the data to determine the effect of inoculation on seedling emergence.
k) Results illustrated that cowpea, groundnut, and soybean seeds inoculated with strain SVRGM/DBT2/03/2020 enhanced seed emergence by 32 to 36% compared to the control.

### Example: 5—Effect of Bacterial Strain CGAPGPBBS-048 Inoculation on Cowpea, Groundnut, and Soybean Yield in Field Trials During the Rainy and Dry Season

i. The trial was conducted in the 2016 rainy season from July to December 2016 and in 2017 dry season from January to June 2017 at two different locations using strain CGAPGPBBS-048 with three crops cowpea, groundnut, and soybean.
ii. The field trials were planted through a commercial seed planter, and foliar bacterial treatment was applied using 2 a handheld sprayer of 250-300 litters per hectare.
iii. All field trials were conducted in the randomized complete block design (RCBD), each crop trials were a size of 10 acres, with bacterial treatment along with two kinds of control (chemical fertilizer control and untreated control), replicated three times. The treatments were designed to evaluate the bacterial ability to enhance crop yield.
iv. Crops were harvested at maturity, seed was collected and cleaned, and yield was measured.
v. Results illustrated that cowpea, groundnut, and soybean yield significantly enhanced as compared to controls.

TABLE 2
Result of example 5 Effect of PGPR Inoculation on Cowpea, groundnut,
and Soybean Yield in Field Trials during the Rainy and dry season.

| | Location 1 | | | Location 2 | | |
|---|---|---|---|---|---|---|
| Treatments | Cowpea | Groundnut | Soybean | Cowpea | Groundnut | Soybean |

Rainy season

| | | | | | | |
|---|---|---|---|---|---|---|
| CGAPGPBBS- | 1544.96 ± | 1908.90 ± | 1777.09 ± | 1330.56 ± | 1765.86 ± | 1860.98 ± |
| 048 | 15.22a | 24.95a | 5.89a | 17.39a | 11.66a | 24.33a |
| Chemical | 1349.85 ± | 1241.28 ± | 1437.81 ± | 1044.90 ± | 1525.79 ± | 1678.17 ± |
| control | 13.30b | 16.22b | 4.77b | 13.66b | 10.07b | 21.94b |
| Untreated | 737.86 ± | 1025.49 ± | 1210.08 ± | 949.36 ± | 1263.00 ± | 1069.76 ± |
| control | 7.27c | 13.40c | 4.01c | 12.41c | 8.34c | 13.98c |

Dry season

| | | | | | | |
|---|---|---|---|---|---|---|
| CGAPGPBBS- | 2565.86 ± | 1769.97 ± | 2556.92 ± | 2283.60 ± | 1736.59 ± | 2299.16 ± |
| 048 | 25.28a | 23.14a | 8.48a | 29.85a | 11.46a | 30.05a |
| Chemical | 2166.91 ± | 1564.29 ± | 1926.26 ± | 2021.79 ± | 1472.40 ± | 1903.76 ± |
| control | 21.35b | 20.45b | 6.39b | 26.43b | 9.72b | 24.88b |
| Untreated | 1962.64 ± | 1551.07 ± | 1654.75 ± | 1670.36 ± | 1226.87 ± | 1484.07 ± |
| control | 19.34c | 20.27b | 5.49c | 21.83c | 8.10c | 19.40c |

Columns marked with the same alphabetical letter(s) within comparable means (n = 3) in the same column do not differ significantly using the revised least significant difference (LSD) test at p = 0.05 levels;
Mean ± standard deviation

### Example: 6—Identification Process of Rhizobacterial Strain

a) The bacterial strain CGAPGPBBS-048 was grown on nutrient agar media for 24 hours. Then the bacterial DNA was extracted.
b) The partial 16S rRNA gene was amplified, then subjected to cycle sequencing through MACROGEN, Korea.
c) The amplified product was sequenced using the forward sequencing reaction mix. The DNA sequence was searched for homology using BLAST search engine at NCBI site (ncbi.nlm.nih.gov) and FASTA (ebi.ac.uk).
d) In the FASTA homology search showed similarity towards the Bacillus Cereus, and the strain obtained from NCBI was designated as KY495213.

Example: 7—Commercial Exploitation (viability and phytotoxicity): The Bacillus strain CGAPGPBBS-048 may demonstrate the ability to produce indole acetic acid in higher pH (alkaline soils) and positively influence the germination and yield of diverse crop varieties. Although some rhizospheric microbes may possess advantageous traits for other crops, the presence of specific bacterial populations in the rhizosphere, without modification or supplementation, is primarily may be determined by the availability of substrates, prevailing environmental conditions (such as soil moisture, pH, and organic matter content), and the competition among different microbial communities. These beneficial microbes may establish colonization on plant roots, enhancing nutrient absorption, synthesizing growth hormones, and providing protection against diseases, thereby promoting overall plant growth and health.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

We claim:

1. A carrier-based agricultural biofertilizer composition, comprising:
an isolated Bacillus Cereus strain CGAPGPBBS-048 having the deposit accession number KY495213;
an agriculturally acceptable liquid formulation including a stabilizer, whereby the stabilizer provides stability and support for at least one of the Bacillus species, thereby enhancing the growth and overall health of the plants.

2. The composition, as claimed in claim 1, wherein the Bacillus Cereus strain CGAPGPBBS-048 is capable of producing indole acetic acid in higher pH conditions by expressing a gene encoding an indole acetic acid-producing enzyme.

3. The composition as claimed in claim 1, wherein the Bacillus Cereus strain CGAPGPBBS-048 was isolated from rhizosphere soil of Yola, Nigeria.

4. The composition as claimed in claim 1, wherein the Bacillus Cereus strain CGAPGPBBS-048 was isolated by a serial dilution technique.

5. The composition as claimed in claim 1, the Bacillus Cereus strain CGAPGPBBS-048 has the ability to stimulate plant growth through the production of phytohormones, including indoleacetic acid, thereby regulating plant growth, improving root development, and enhancing overall plant vigor.

6. A method for enhancing soil fertility using a carrier-based agricultural biofertilizer composition, comprising the steps of:
preparing a nutrient medium containing optimized sources of carbon, nitrogen, and phosphate, conducive to bacteria growth;
inoculating the nutrient medium with a pure culture of a Bacillus Cereus strain CGAPGPBBS-048, characterized by deposit accession number KY495213;
fermenting the inoculated medium under precisely controlled conditions until the broth attains a concentration of 3.0×10\hat{ }9 CFU/mL;
introducing polyvinyl pyrrolidone (PVP) into the broth at a concentration of 0.5%;
producing a bacterial formulation by culminating the fermentation process;
diluting the bacterial formulation with precision to attain a targeted microbial concentration of 3×10\hat{ }8 CFU/mL; and
systematically applying the meticulously prepared bacterial formulation to the soil using appropriate methods to ensure even and comprehensive distribution.

7. The method as claimed in claim 6, wherein the nutrient medium comprises carbon sources selected from the group consisting of sucrose and glucose, whereby the carbon sources provide optimal conditions for bacterial growth and nutrient assimilation.

8. The method as claimed in claim 6, wherein the nutrient medium comprises nitrogen sources selected from the group consisting of ammonium sulfate, urea, and peptone, whereby the nitrogen sources provide optimal conditions for bacterial growth and nutrient assimilation.

9. The method as claimed in claim 6, wherein the step of fermenting conditions is carried out under controlled conditions including temperature, pH, and oxygen levels, to thereby promote optimal bacterial growth and metabolic activity.

10. The method as claimed in claim 6, wherein the step of systematically applying the meticulously prepared bacterial formulation to soil is performed using sprinkler irrigation, spray application, drone-based application, soil mixing techniques, seed deepening methods, plant root deepening methods, or any combination thereof.
